(19) United States
(12) Patent Application Publication
(10) Pub. No.: US 2024/0301255 A1
(43) Pub. Date: Sep. 12, 2024

(54) EYEWEAR SUPPORT ADHESIVE

(71) Applicant: Neil GAY, Terrigal (AU)

(72) Inventor: Neil GAY, Terrigal (AU)

(21) Appl. No.: 18/262,713

(22) PCT Filed: Feb. 9, 2022
(86) PCT No.: PCT/AU2022/050081

§ 371 Date: Jul. 24, 2023

(30) Foreign Application Priority Data
Feb. 10, 2021 (AU) 2021900314

(51) Int. Cl. C09J 133/26; C09J 5/00; C09J 133/04; G02C 5/12

(52) U.S. Cl. CPC C09J 133/26; C09J 5/00; C09J 133/04; C09J 2433/00; G02C 5/12

(57) ABSTRACT

Eyewear support adhesive which is applied directly to contact surfaces of ophthalmic supports of eyewear (such as nose pads) to bond to the skin and to prevent or reduce the likelihood of the eyewear sliding down the bridge of the nose comprises a resin within a carrier which evaporates to leave a tacky residue of resin to form a bond with the skin. The resin comprises Acrylates/Octylacrylamide Copolymer which was found by trial and experimentation to have superior perspirant resistant holding properties when used on nose pads applied to the skin, thereby negating the need for antiperspirant additive.

### FIELD OF THE INVENTION

This invention relates to eyewear support adhesive which can be applied to eyewear supports, such as nose pads, to maintain support.

### BACKGROUND OF THE INVENTION

Slipping of eyewear down the nose is a common problem for which many solutions have been proposed, including heating and bending distal ends eyewear arms downwardly, applying adhesive sheaths to distal ends of the eyewear arms and the like.

U.S. Pat. No. 4,251,302 A (LEONARD et al.) 17 Feb. 1981 proposes a pressure-sensitive adhesive which is applied to eyewear ophthalmic supports in a diluent which dries at ambient conditions to form a bond with the skin.

As perspiration can weaken the bond, LEONARD et al. further suggests using an antiperspirant (such as aluminum clorohydrate) to beneficially address loss of adherence when the wearer of the eyewear is perspiring.

### SUMMARY OF THE DISCLOSURE

There is provided herein eyewear support adhesive which is applied directly to contact surfaces of ophthalmic supports of eyewear (such as nose pads) to bond to the skin and to prevent or reduce the likelihood of the eyewear sliding down the bridge of the nose.

The adhesive comprises a resin within a carrier which evaporates to leave a tacky residue of resin to form a bond with the skin.

The resin comprises Acrylates/Octylacrylamide Copolymer. Acrylates/Octylacrylamide Copolymer is a copolymer of octylacrylamide and one or more monomers consisting of acrylic acid, methacrylic acid or one of their simple esters normally applied in aerosols, pump hair sprays and styling aids. However, Acrylates/Octylacrylamide Copolymer was found by trial and experimentation to surprisingly confer superior perspirant resistant holding properties when applied to eyewear nose pads applied to the skin, thereby negating the need for antiperspirant additive taught by LEONARD et al. to mitigate against perspiration, especially in sporting activities.

The adhesive may comprise a thickening agent assisting the beading of the adhesive on the nose pads when applied to the nose pads with a dispensing applicator (such as a squeeze bottle or adsorbent tip).

The quantity of resin may be adjusted according to a desirous tackiness characteristic (i.e., strong or weak adhesion). Furthermore, the adhesive may comprise an adhesion retardant, wherein the ratio thereof to the resin may be adjusted to control the tackiness of the adhesive, such as whether the adhesive is to be used for sports application which would require greater adhesion as compared to for reading application for example.

The adhesive may further comprise pH neutralising agents, humectants and emollients.

The disclosure herein provides certain chemicals and compositions and specific ratios thereof which have been experimentally derived by the present applicant and methods of making the present adhesive and for the use thereof.

Other aspects of the invention are also disclosed.

### DESCRIPTION OF EMBODIMENTS

Eyewear support adhesive is applied as a coating on ophthalmic support contact surfaces of eyewear, such as nose pads, to prevent the eyewear slipping down from the bridge of the nose.

The adhesive comprises an evaporative carrier and a resin comprising Acrylates/Octylacrylamide Copolymer.

The evaporative carrier preferably comprises alcohol (for swifter evaporation as compared to water for example). The alcohol is preferably ethanol.

The ethanol may be between 60%-80% by weight, preferably 76% by weight. The ethanol may be 95PGF2 perfume grade ethanol or Bio-Ethanol 95%.

The Acrylates/Octylacrylamide Copolymer and may be between 10% at 20% by weight, preferably 15% by weight.

The presented weight of resin may be varied depending on a desirous tackiness characteristic of the adhesive.

The adhesive may further comprise a thickening agent which may comprise hydroxypropyl cellulose. The hydroxypropyl cellulose may be approximately 1% by weight.

The adhesive may further comprise a pH neutralising agent such as aminomethyl propanol which may be between 2-8% by weight, preferably 4% by weight.

The adhesive may further comprise a humectant which may be glycerin or lanolin at between 1%-5% by weight, preferably approximately 2% by weight.

The adhesive may further comprise an emollient which may comprise cetyl acetate and acetylated lanolin alcohol at between 1-5% by weight, preferably approximately 2% by weight.

The adhesive may further comprise an adhesion retardant which may be magnesium carbonate. The magnesium carbonate may be between 1%-10% by weight, preferably approximately 5% by weight. However, the ratio of magnesium carbonate to resin may be varied depending on the desirous tackiness characteristic of the adhesive. For example, increasing the ratio of magnesium carbonate to resin to more than 5% may reduce the tackiness, thereby being more suitable for reading applications and the like whereas reducing the ratio of magnesium carbonate to resin to less than 5% may increase the tackiness, thereby be more suitable for sporting applications and the like.

Making the adhesive may comprise dispersing and mixing the hydroxypropylcellulose in the ethanol until lump free and then allowed to thicken.

The Acrylates/Octylacrylamide Copolymer may then be added and mixed until dissolved.

Thereafter the aminomethyl propanol may be mixed in until uniform and wherein the batch becomes somewhat thinner.

Thereafter the glycerin and cetyl acetate and acetylated lanolin alcohol may be mixed in until uniform.

The adhesive may be provided in a dispenser comprising a reservoir soaking an applicator tip with the adhesive. The reservoir may be a small squeeze bottle connected to the applicator tip by a delivery tube. As such, the reservoir may be squeezed to soak the applicator tip with the adhesive which can then be wiped across the surfaces of the eyewear nose pads.

The carrier may be allowed to evaporate somewhat whereafter the eyewear may be applied to the bridge of the nose wherein the resin holds the eyewear nose pads to the skin at the bridge of the nose. The adhesion retardant may control the tackiness thereof.

Removing the eyewear may comprise lifting the eyewear at the rear of the arms thereof to tilt the eyewear forwardly to peel, rather than pull, the eyewear nose pads from the bridge of the nose.

Whereas the embodiments provided herein have described the application of the adhesive to the eyewear nose pads in accordance with a preferred embodiment, in alternative embodiments, the adhesive may be applied to the inner surfaces of distal ends of the eyewear arms additionally or alternatively.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

The term “approximately” or similar as used herein should be construed as being within 10% of the value stated unless otherwise indicated.

1. An adhesive for eyewear support, comprising:
an evaporative carrier; and
a resin comprising Acrylates/Octylacrylamide Copolymer.

2. The adhesive as claimed in claim 1, wherein the adhesive does not comprise an antiperspirant.

3. The adhesive as claimed in claim 1, wherein the evaporative carrier comprises alcohol.

4. The adhesive as claimed in claim 3, wherein the alcohol comprises ethanol.

5. The adhesive as claimed in claim 4, wherein the adhesive comprises the ethanol at between 60%-80% by weight.

6. The adhesive as claimed in claim 4, wherein the ethanol is selected from 95PGF2 perfume grade ethanol and Bio-Ethanol 95.

7. The adhesive as claimed in claim 1, wherein the adhesive comprises the resin at between 10-20% by weight.

8. The adhesive as claimed in claim 1, further comprising a thickening agent.

9. The adhesive as claimed in claim 8, wherein the thickening agent comprises hydroxypropyl cellulose.

10. The adhesive as claimed in claim 8, wherein the adhesive comprises the thickening agent at approximately 1% by weight.

11. The adhesive as claimed in claim 1, further comprising a pH neutralising agent.

12. The adhesive as claimed in claim 11, wherein the pH neutralising agent comprises aminomethyl propanol.

13. The adhesive as claimed in claim 11, wherein the adhesive comprises the pH neutralising agent at between 2-8% by weight.

14. The adhesive as claimed in claim 1, further comprising a humectant.

15. The adhesive as claimed in claim 14, wherein the humectant comprises glycerin.

16. The adhesive as claimed in claim 14, wherein the adhesive comprises the humectant at between 1%-5% by weight.

17. The adhesive as claimed in claim 1, further comprising an emollient.

18. The adhesive as claimed in claim 17, wherein the emollient comprises cetyl acetate and acetylated lanolin alcohol

19. The adhesive as claimed in claim 17, wherein the adhesive comprises the emolument at between 1%-5% by weight.

20. The adhesive as claimed in claim 1, further comprising an adhesion retardant.

21. The adhesive as claimed in claim 20, wherein the adhesion retardant comprises magnesium carbonate.

22. The adhesive as claimed in claim 21, wherein the adhesive comprises the adhesion retardant at between 1-10% by weight.

23. The adhesive as claimed in claim 20, wherein a ratio of adhesion retardant to resin is determined according to a desirous tackiness characteristic of the adhesive.

24. A method comprising applying the adhesive as claimed in claim 1 to ophthalmic supports of eyewear.

25. The method as claimed in claim 24, wherein the method further comprises cleaning the ophthalmic supports with alcohol prior application of the adhesive.

26. The method as claimed in claim 25, wherein the method further comprises allowing the evaporative carrier to evaporate somewhat prior placing the ophthalmic supports.

27. A method of removing eyewear having nose pads coated with the adhesive as claimed in claim 1, the method comprising lifting the eyewear rearwardly by the arms thereof such that the nose pads peel from the skin of the bridge of the nose.

28. A method of making the adhesive as claimed in claim 1, the method comprising dispersing a thickening agent in the evaporative carrier and mixing the thickening agent and evaporative carrier until free of lumps.

29. The method as claimed in claim 28, further comprising allowing the mixture of the thickening agent and the carrier to thicken.

30. The method as claimed in claim 29, further comprising adding the resin until dissolved.

31. The method as claimed in claim 30, further comprising adding a pH neutralising agent after adding the resin.

32. The method as claimed in claim 31, further comprising adding a humectant and emollient after adding the pH neutralising agent.

33. The method as claimed in claim 30, further comprising adding an adhesion retardant.

34. The method as claimed in claim 33, wherein adding the adhesion retardant further comprises adding an amount of the adhesion retardant according to a desired tackiness characteristic of the adhesive.

35. The method as claimed in claim 30, wherein adding the resin comprises adding an amount of the resin according to a desired tackiness characteristic of the adhesive.
